(19) United States
(12) Patent Application Publication
(10) Pub. No.: US 2013/0059324 A1
(43) Pub. Date: Mar. 7, 2013

(54) SYSTEMS AND METHODS FOR MECHANICALLY STRAINED CELL CULTURE

(71) Applicants: Gurusingham Sitta Sittampalam, Lenexa, KS (US); Victor Sanjit Nirmalanandhan, Prairie Village, KS (US)

(72) Inventors: Gurusingham Sitta Sittampalam, Lenexa, KS (US); Victor Sanjit Nirmalanandhan, Prairie Village, KS (US)

(73) Assignee: THE UNIVERSITY OF KANSAS, Lawrence, KS (US)

(21) Appl. No.: 13/696,953

(22) PCT Filed: May 11, 2011
(86) PCT No.: PCT/US11/36035

§ 371 Date: Nov. 8, 2012

Related U.S. Application Data
(60) Provisional application No. 61/333,707, filed on May 11, 2010.

(51) Int. Cl. C12Q 1/02; C12M 1/34

(52) U.S. Cl. 435/29; 435/288.4

(57) ABSTRACT

A method for in vitro drug screening can include: providing one or more cells in vitro; introducing a chemical to the one or more cells; introducing a mechanical strain to the one or more cells in the presence of the chemical; and determining whether or not the chemical has bioactivity with respect to the one or more cells under the mechanical strain. The mechanical strain can be implemented with a well plate defining a plurality of cell culture wells and having one or more flexible substrates associated with the well plate so that each cell culture well has a fluid tight flexible cell culture substrate. A dynamic in vitro screening device can include: a top plate defining one or more cell culture wells; one or more flexible cell culture substrates operably coupled with the top plate to form fluid tight cell culture well bottoms; an a pin plate under the top plate with the one or more flexible cell culture substrates therebetween, the pin plate having one or more pin members associated with the one or more cell culture wells.

### PRIORITY CLAIM

This patent application claims the benefit of U.S. 61/333,707, filed on May 11, 2010, which provisional patent application is incorporated herein by specific reference in its entirety.

### BACKGROUND

The current drug discovery paradigm for testing bioactivity, such as for chemotherapeutic agents that have activity against cancer, is inefficient and expensive. In part, the standard drug discovery model is not representative of the real life conditions and dynamics of in vivo tissues. Currently, the most common drug discovery paradigm uses static 2-dimensional cellular models, such as in monolayer cell cultures, for drug screening. The cell cultures usually are left undisturbed. Such static 2-dimensional cellular models are not representative of the living conditions of cells and tissues experienced in vivo, and do not reliably translate the pharmacological activity of potential drugs from testing to in vivo applications. These 2-dimensional cellular models do not incorporate the real life conditions of the cells or tissues and do not provide meaningful information for the dynamics experienced by living cells and tissues. Unfortunately, nearly 90% of the lead chemical hits that are identified by the current drug discovery paradigm that uses cell culture monolayers in vitro screening systems fail in preclinical studies and clinical trials. Therefore, drug discovery techniques that more accurately simulate the real life living conditions of cells and tissues need to be explored. See: Petski, G. A. (2010) The Devil's in the Details. Genome Biol. 11, 117.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIGS. 1A-1B include schematic representations of an exploded view (FIG. 1A) and side view (FIG. 1B) of an embodiment of a plate in accordance with the present invention.

FIGS. 2A-2C include schematic representations of embodiments of plate systems in accordance with the present invention.

FIGS. 3A-3C include different views of a schematic representation of an embodiment of a top plate in accordance with the present invention.

FIG. 4A-4C include different views of a schematic representation of an embodiment of a frame that couples a flexible membrane sheet and/or the pin plate to the top plate of FIGS. 3A-3C.

FIGS. 5A-5B include different views of a schematic representation of an embodiment of a pin plate in accordance with the present invention.

FIGS. 5C and 5D include different views of a schematic representation of an embodiment of a pin of the pin plate of FIGS. 5A-5B.

FIG. 5E includes a schematic representation of optional embodiments of a movable pin that is movable in a pin plate that has a pin hole in accordance with the present invention.

FIG. 6 includes a schematic representation of an embodiment of a computing system that can be used to control a plate system in order to perform the mechanically strained cell culture methods and drug screening methods in accordance with the present invention.

FIG. 7 includes a schematic representation of an embodiment of a system for implementing mechanically strained cell culture methods and/or drug screening with an embodiment of a plate system in accordance with the present invention.

FIG. 8 includes a schematic representation of the experimental design depicting 6 treatments and the corresponding mathematical models. As shown, in Treatment 6, no forces were applied during the 1st day, forces were applied for the next 5 days, the drug was added after 3 days and the number of live cells was counted at the end of the 6th day. Analogous interpretations can be given for the other treatments. Dotted lines represent the drug exposure and the dashed lines represent the simulation of in vivo forces.

FIG. 9A shows a side view of an embodiment of a well of a plate having a flexible membrane sheet bottom that is un-stretched.

FIG. 9B shows a side view of an embodiment of a well of a plate having a flexible membrane sheet bottom that is stretched.

FIG. 9C shows a simulation of an applied mechanical force profile showing the changes in strain from 0 to 20% and back to 0% during the first 2 seconds of the cycle, followed by 2 seconds of rest at 0% strain.

FIG. 10A includes a graph that illustrates data of growth of A549 cells in Treatments 2, 3, 5 and 6, and show the changes in growth rates in response to the Cisplatin treatment and/or simulated forces.

FIG. 10B includes a graph that illustrates data for an A549 experiment showing the effectiveness of Cisplatin in both the absence and presence of mechanical forces. Each treatment is represented as median viable cells per well ±SEM. The differences between the treatments are shown as percent reductions in viable cell number. The percent reduction in viable cells between Treatments 2 and 3 is defined as the effectiveness, E, of the drug in the absence of the forces. The percent reduction in viable cells between Treatments 2 and 5 captures the changes in the live cell count produced by forces alone. The percent reduction in viable cells between Treatments 5 and 6 represents the changes in live cell count due to the cytotoxicity (or effectiveness, E_{f}) of the drug in the presence of forces.

FIG. 11 includes: microphotograph images of A549 cells after 72 hours with and without mechanical forces (Panel A); microphotographs of NCI-H358 cells after 72 hours (Panel B); and the NCI-H358 cells after 144 with and without mechanical forces (Panel C).

FIGS. 12A-12B include dose-response graphs that illustrate change in absorbance of H358 cells grown as 3D tissues in collagen with and without mechanical forces in response to increase drug concentration.

FIGS. 13A-13B include graphs that illustrate cell viability counts of A549 cells and H358 cells over time with or without mechanical forces.

All elements of the figures are arranged in accordance with or representative of at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present invention relates to compositions, devices, systems and methods for performing drug screening techniques that utilize mechanical motion to impart strain to cell cultures in the presence of a potential drug. The mechanical motion can be imparted to two-dimensional (2D) and three-dimensional (3D) cell cultures or tissues while the potential drugs are screened with cells concurrently experiencing stretching, compression, or other movement that mimic in vivo conditions. As such, the invention can relate to 2D and 3D cell culture drug discovery assays, 2D and 3D tissue models for drug discovery assays, 2D and 3D dynamic cell culture environments for drug discovery, 2D and 3D mechanical stimulation of cells in drug discovery, and improved devices and systems for implementing cell culture drug discovery assays under mechanical motion. Also, the invention can include bioreactors for high throughput screening (HTS), and devices and systems for cell culture and tissue modulated drug discovery screening assays.

Additionally, the invention can provide a more physiologically relevant screening system for drug discovery. The physiologically relevant screening can mimic the motion, movement, strain, elongation of any tissue or organ. A physiological strain simulation can be conducted by a computing system controlling the cell motion, movement, position, stretch, or the like during drug screening. The computing system can include instructions that cause the cell culture system to impart physiologically relevant strain to the cells by stretching a cell culture as described herein.

The invention includes an improvement to existing drug screening paradigms that only use screening technologies where cells are static or rarely moved. Rarely are drugs used on patients in whom the organs and tissues remain static. As such, the present invention provides for a drug screening paradigm that imparts strain to cells in a manner to attempt to simulate physiological motion and rest. The new mechanically strained drug screening paradigm can provide a new and improved approach for screening various types of drugs for specific receptor, cell, organ or tissue targets.

For example, a mechanical straining drug screening device, system, and method can be useful for modulating lung tissue cultures by stretching the cultures in various stretch patterns. The stretching can cause the cells in the culture to be under mechanical strain so that a potential drug (e.g., anti-lung cancer agents) can be studied in a dynamic system of movement and mechanical strain with optional periods of relaxation.

The improvement can also include comparing drug screening results from the traditional static cell culture model with the new mechanically strained cell culture model to provide a physiologically relevant model. As such, the drug screening can compare data obtained with the standard static cell culture models with the data from the models in the presence of simulated in vivo forces, such as with forces and movement that mimic respiration. This approach can provide an enhanced simulation of physiologically relevant motion strain and mechanical strains, which can be better predictive of in vivo activity and therefore provide data more comparable to pre-clinical animal models and clinical outcomes for drug candidates.

The simulated mechanical strains can include any mechanical forces that mimic in vivo conditions or in vivo strains that are imparted to cells or organs. Such mechanical strains can include stretching or compression in any dimension or direction under any load with any acceleration and any velocity. Also, the mechanical strains can be implemented in a cyclic manner so as to have cyclic stretching and/or cyclic compression, which may also include a mechanical strain environment that stretches the cells and then compresses the cells in any dimension or direction, or compresses the cells then stretches the cells in any dimension or direction. Random patterns as well as computer-generated patterns of motion and rest can be used to simulate various activities.

While lung cancer and lung cancer drug candidates are described herein, the present technology can be applied to substantially any type of disease of any organ or tissues as well as for screening any type of drug for any indication. Lung cancer is used herein as a representative disease state for which drug candidates can be screened using the technology described herein. Other types of cells can be used in place of the lung cancer cells described herein. Additionally, although this disclosure focuses discussion on lung cancer drug discovery, the descriptions of the invention can also be useful for general drug screening or chemical-induced cell function studies by imparting mechanical forces on the cells during the screening.

The in vitro chemical screening under mechanical strain can be used for all types of cancer (e.g., colon and bladder cancer) and other types of diseases (e.g., musculoskeletal and cardiac diseases). The processes of in vitro screening under mechanical strain can be used to screen various types of molecules in the presence of simulated in vivo mechanical forces in pathologies associated with many organ systems (e.g., musculoskeletal, gastrointestinal, cardiac and respiratory systems). Any type of chemical or combination of chemicals can be used, however, the systems and methods can be well-suited to drug discovery or development.

Although effects of the in vivo forces have been studied on different cell lines including lung and colon cancer cell lines for other purposes, no studies have tested the potency and efficacy of drugs on cells in culture in the presence of these forces. Testing cancer drug candidates in cell cultures and/or tissue models in the presence of simulated in vivo forces or mechanical forces similar to those experienced in normal physiological conditions (e.g. respiration or digestion) can provide data that is more physiologically relevant and potentially more predictive towards improving the translation of in vitro drug activity to clinical outcomes of anti-cancer drug candidates. Therefore, in vitro drug screening in the presence of mechanical forces can now be used to identify drug candidates that demonstrate improved and superior activity against lung cancer cells as well as substantially any other type of cell or disease state.

During the drug development process, lead drug candidates identified with in vitro screening under mechanical strain can be then tested in preclinical animal studies followed by clinical trials. The results of the in vitro screening under mechanical strain can be better translated to preclinical studies over screening that does not involve the use of mechanical strains, and therefore it is likely that a higher percentage of clinical trials can be successful. Since the typical in vitro screening systems utilize cell cultures that are static monolayers and studied to assess biological activity of selected molecules to determine suitability to be tested in animal models, nearly 90% of the lead candidates identified fail clinical trials. This failure is likely an indicator that the current motion and strain static biochemical and biological assays cannot reliably translate biological or pharmacological activity of a chemical substance.

The large percentage of lead drug candidates with high in vitro potency in traditional screening assays (e.g., without mechanical strain) that then fail to show efficacy in animal models may be due to the static nature of the experiment. In part, this may be due to the signaling pathways in non-strained cell culture models not representing or adequately simulating cellular behavior in the tissue architecture in animals in rest or in motion. The morphology, differentiation, cell-cell and cell-extracellular matrix interactions of cells grown on rigid cell culture substrates are remarkably different from those of the cells grown in more physiological-relevant mechanically strained environments.

Many organs experience constant changes in physiological conditions (e.g., 3D architecture, mechanical strains, etc.) during development of disease pathology. Failure to simulate the effect of these conditions during in vitro screening phase of drug discovery can result in selecting molecules that will not show efficacy in vivo. Three dimensional culture systems alone are not the only physiologically relevant condition with respect to some tissues and organs. Cells in tissues, such as lung, musculoskeletal systems, and the alimentary canal, experience mechanical forces in vivo through normal respiration or distention and compression of the colon through peristalsis. These in vivo mechanical forces have been shown to impact signal transduction pathways affecting cell biology and potentially the mechanisms through which drugs function. The invention accounts for the in vivo mechanical forces that a cell may experience in its respective microenvironment (e.g. lungs, colon and bladder). Thus, the invention includes using in vitro cell cultures that are impacted by mechanical forces for drug screening.

A new mechanically straining in vitro screening device and system can be beneficial for improvements with in vitro screening under mechanical strain as described herein. The mechanically straining in vitro screening device and system can include a cell culture plate with any number of sample wells. The cell culture plate can be a high throughput plate or any other type of plate, and adapted to have mechanical straining functionality. The mechanical straining functionality can be achieved by mechanically stretching the cells as described herein, particularly with the cylindrical pins with rounded heads that provide bi-axial stretching to the cells. The mechanically straining in vitro screening device and system can be designed for drug screening applications and to be compatible with standard plate readers or imaging devices.

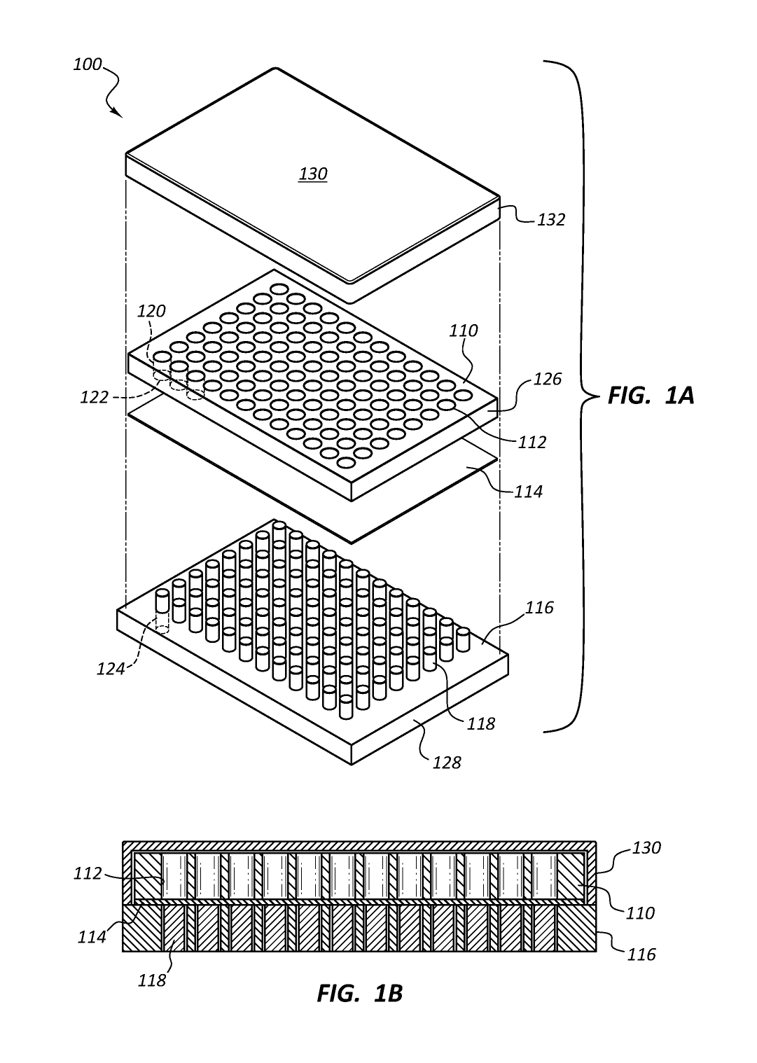

FIG. 1A includes an exploded view of a plate 100 configured in accordance with the present technology which allows for cell culturing to be conducted under mechanical strain. The plate 100 as shown includes a top plate 110 having a plurality of individual sample wells 112. The top plate 110 is shown to have 96 sample wells 112, however, any other suitable number can be used. 96 wells can be beneficial as it allows for the plate 100 to be usable with many standard plate readers, automated liquid dispensing devices, and HTS robots according to SBS plate standards, because 96 wells is a common number for cell culture assay plates; however, substantially any reasonable number of wells 112 can be included in the top plate 110.

Below the top plate 110 is a flexible membrane sheet 114 that is coupled to the top plate 110. The flexible membrane sheet 114 is attached to the top plate 110 in a manner that each individual well 112 is sealed. That is, each well 112 can include a well chamber 120 that has a bottom surface 122 that is sealed by the flexible membrane sheet 114. As such, the individual wells 112 are fluid tight at the bottom and contents thereof cannot pass from one well 112 to another well 112 by passing from the bottom of the well chamber 120. This can be achieved by adhering the sheet to the plate around each of the wells to form the chamber 122 with a fluid tight flexible membrane sheet bottom 122.

Below the flexible membrane sheet 114 is a pin plate 116 that has a pin 118 for each well 112 of the top plate 110. In one configuration, the pin plate 116 is affixed to each pin 118 so that the pin plate 116 and pins move together and are rigid with respect to each other.

In another configuration, the pin plate 116 can include a pin hole 124 for each of the pins 118. The pin hole 124 can be adapted to slideably receive the pin 118 when the pin 118 drops and allows for a pushing member (not shown) to contact the bottom of the pin 118 in order to push the pin 118 upwards so as to contact the flexible membrane sheet 114 at the bottom surface 112 of the well chamber 120. As such, the pin hole 124 is substantially configured as an aperture or tunnel that passes through the pin plate 116. The pin hole 124 can have no bottom so that a pushing member can push the pin 118 upwards and allow. The pin 118 and pin hole 124 can be adapted with ridges, flanges, or the like to retain the pin 118. The pins 118 and pin holes 124 can be jointly or separately coupled to a pneumatic system to lift or prop the pins in the pin 118 holes 124.

This allows the membrane bottoms 122 to be moved together or independently. Accordingly, the pins 118 can move, compress, or stretch the membrane bottom 122 for each of the wells 112 independently to simultaneously. Also, membrane bottoms 122 of the wells 112 of various rows or columns can be mechanically strained together and/or differently from other rows or columns or wells, where the pin plate and system can be configured accordingly.

The plate 100 can also include a lid 130 that is adapted to be received over the top plate 110. The lid 130 can be configured as any standard well plate lid. The lid 130 can be flat and lay over the top plate 110, or the lid 130 can include edges 132 adapted to be received over the edges 126 of the top plate 110, and optionally over the edges 128 of the pin plate 116. The lid 130 may also include features that can cover the individual well top openings, such as plug members to plug the well hole openings jointly or separately.

While not shown, the lid 130 can include plugs that act to fluidly plug the individual wells. This can be useful because, as the pin 118 is pulled down, the plugged well can have the membrane bottom 122 pulled downward to deliver a mechanical strain. Cells can still experience tensile stretching and mechanical in this configuration.

The plate can be configured to apply compressive forces to the cells. In order to achieve compressive forces, the method can include pushing the pins upward as is described herein, and to simultaneously prevent the upward deformation of the membrane by having a plug in the top opening of the well. For example, this strategy can apply forces to cells from the bottom of the membrane using the loading pins and simultaneously applying forces from the top of the membrane by using stationary plugs.

In one embodiment, the pins can be associated with a lubricant. The lubricant can be used to reduce the friction between the pins and the membrane. It can be useful to reduce the friction to prevent the cells from any damage that the heat dissipated as a result of friction may cause. Excessive friction may also damage the membrane and the pins. For example, a layer of silicone lubricant (e.g., Loctite) can be applied to the pins before an experiment is initiated. Any lubricant can be used. The devices, systems, and kits can include the plates as well as the lubricant.

FIG. 1B shows a side view of the lid 130 on the top plate 110, which has the flexible membrane sheet 114 coupled to the bottom of the top plate 110 to seal the bottoms 122 of the wells 112. The pin plate 116 is coupled to the top plate 110 with the flexible membrane sheet 114 therebetween. Also shown are the wells 112 aligning with the pins 118.

In one example, the plate 100 can be fabricated by simply mounting the membrane 114 to a standard well plate 110 frame, such as a 96 well plate frame. As such, the sides of the well plate 110 can be substantially similar as presently available commercial products with the bottom side being the flexible membrane sheet 114 that is flexible at discrete well bottom 122. Also, the membrane 114 can include a frame (not shown) that corresponds with the well plate 110 frame so that there are a number of individual membranes as well bottoms 122. This can be useful with a portion of the membrane 114 being associated with each well as a bottom 122 of the well chamber 120.

The material for the flexible membrane sheet 114 can have the following characteristics: biocompatible for growing cells; able to withstand cyclic stretching; and transparent to facilitate the imaging of cells. Many different types of polymeric membranes can be configured for this purpose.

The shape and the geometry of each pin 118 can be optimized by performing a finite element analysis to ensure the distribution of physiologically relevant strain across the membrane bottom 122 in each well 112. While circular wells and pins are illustrated, the wells and pins can be cooperatively designed to match in substantially any shape and any size. Such shapes can include triangle, square, rectangle, quadrangle, diamond, pentagon, hexagon, octagon, or other polygon or circular or oval shape.

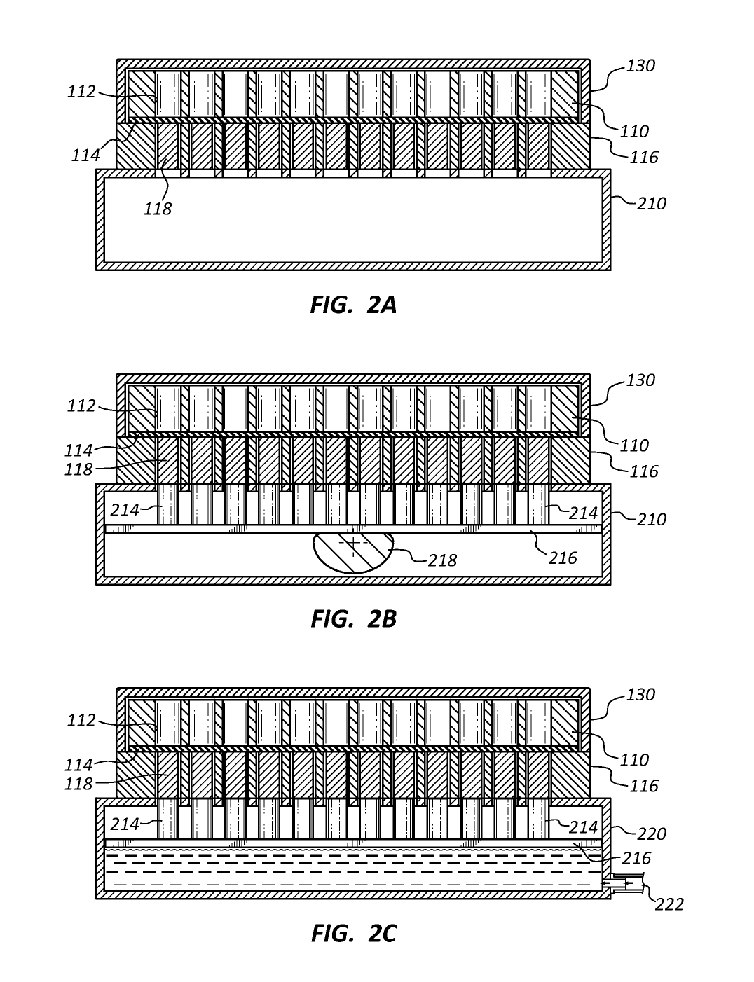

FIG. 2A illustrates a plate system 200 that can include the plate 100 of FIGS. 1A-1B. The plate system 200 further includes a mechanism 210 that is adapted to interact with the pins 118 so as to push the pins 118 against the flexible membrane sheet 114. The pin plate 116 can be located on top of the mechanism 210, and may be removably coupled thereto. It is possible that the pin plate 116 can be affixed to the mechanism 210, otherwise the pin plate 116 can be operably coupled to the mechanism 210 so as to operate as described herein.

In one embodiment, the plate system 200 can be configured to include two major components: a 96-well plate 110 with a flexible membrane sheet 114 and the corresponding pin plate 116 having the pins 118 rigidly or fixedly mounted thereto; and the mechanism 210 configured to actuate the pins 118 against the flexible membrane sheet 114. The pins 118 can be moved up and down against the flexible membrane sheet 114 to provide the mechanical strain to the cells within the wells 112. The movement of the pins 118 against the flexible membrane sheet 114 can be configured to mimic the expanding and contracting lung motion as well as substantially any other physiological movement or movement pattern so that the movement mimics other tissues or organ function.

The mechanism 210 can be configured as a mechanical mechanism that uses mechanical movement of mechanical components in order to move the pins 118 as described herein. Various types of mechanical mechanisms can be configured to provide movement to the pins 118 as described herein, and such mechanical mechanisms can operate to push the pins 118 up and allow for the pins 118 to fall back down or pull the pins 118 back down. Substantially any mechanical mechanism that can perform this function can be implemented into the mechanism 210.

FIG. 2B shows a mechanical mechanism 212 that includes a push member 214 for each pin 118, such that the push member 214 is located below the corresponding pin 118. This configuration can be suitable for a pin plate 116 with individually movable or fixed pins 118. A movable support 216 is located under and supporting the push members 214 so as to be operably coupled thereto. A non-circular shaft 218 is operably coupled to the movable support 216, such that rotation of the non-circular shaft 218 moves the movable support 216 and thereby moves the push members 214 up and down with respect to the pins 218. While the non-circular shaft 218 is shown to have a semi-circular cross-section, other shapes that are not a circle can be used, such as triangles, squares, ovals, or other cross-sectional shapes. The non-circular shaft 218 can be operably coupled to a mechanical drive mechanism (not shown) in order to rotate the non-circular shaft 218 to provide movement to the pins 118.

FIG. 2C is substantially similar to FIG. 2B, except instead of a non-circular shaft 218 operated by a mechanical drive mechanism, the mechanism is a pressurized mechanism 220. The pressurized mechanism 220 can include a fluid port 222 that pushes fluid into the pressurized mechanism 220 to cause the support 216 to move upwards, where the fluid port 222 can also allow fluid to leave the pressurized mechanism 220 to cause the support 216 to move downwards. As such, the flow of fluid into and out from the pressurized mechanism 220 can change the volume of fluid therein so that change of fluid volume moves the support 216.

Accordingly, pneumatics can be used to modulate the pins 118 and thereby move the flexible membrane sheet 114 at select and discrete locations relative to the wells 112. The pneumatics can impart mechanical strains to the cells that are in the wells 112.

The mechanism to actuate the pins 118 can vary. Some non-limiting examples can include: a simple linear actuator; electromagnetic systems; and/or pneumatic systems. In one option, a purely pneumatic system can use pneumatics in place of the pins 118 so that fluid pressure oscillates the membrane bottoms 122. Water, oil, or gas can be used to move the pins. Also, mechanical motors can be used to move the pins 118, such motors can include a stepper motor, cam, or any other linear actuator. Various simple or complex mechanical drive systems can be used to move the pins.

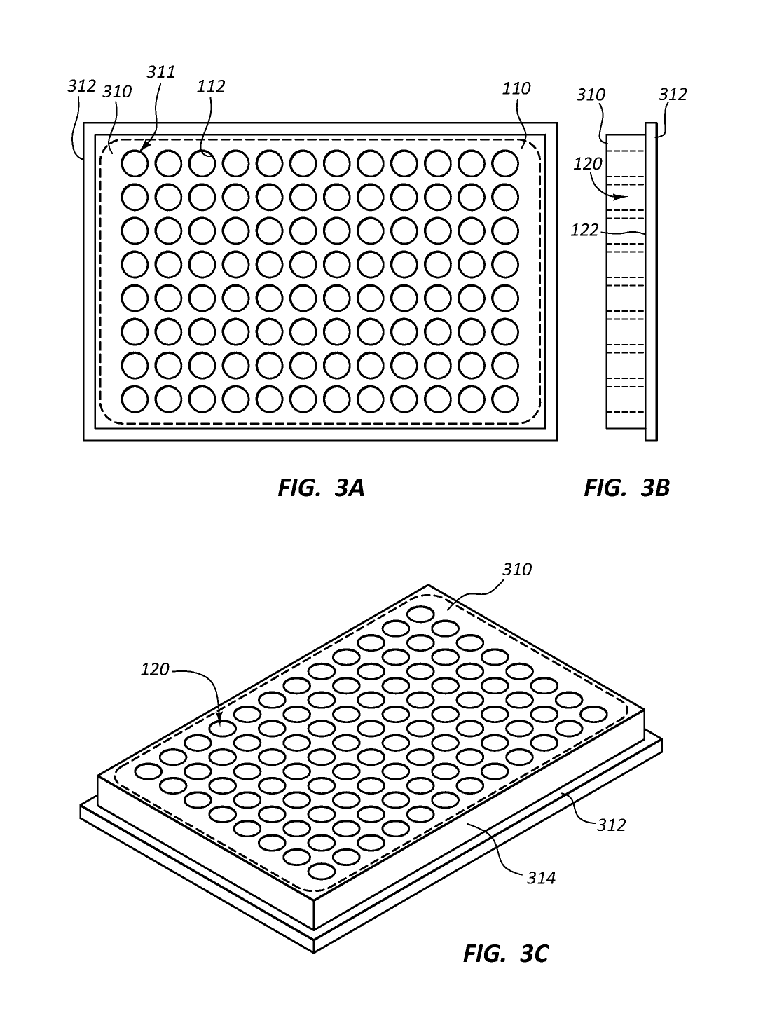

FIG. 3A shows a top view of an embodiment of a top plate 110 having 96 sample wells 112 with well chambers 120 having fluid-tight flexible membrane bottoms 122. FIG. 3B shows a side view and FIG. 3C shows a perspective view. The top plate 110 can include a top surface 310 having well openings 311. A ridge 312 can be located and adapted to receive the lid 130 over the side walls 314. However, the top plate 110 can be configured as any standard or later developed multi-well top plate.

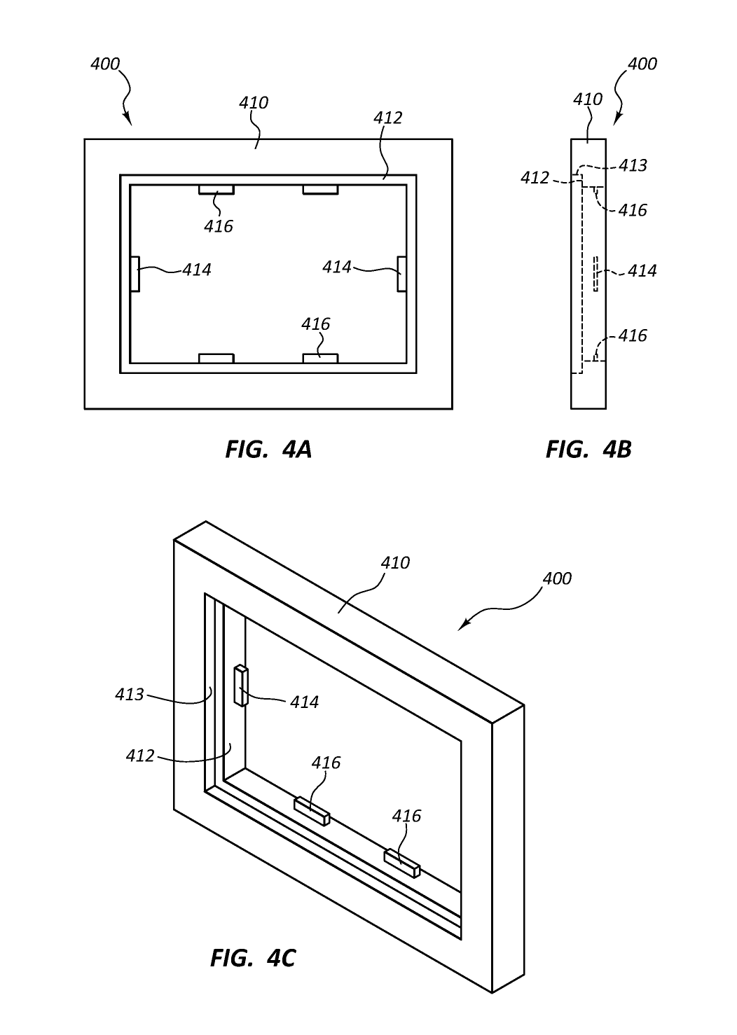

FIG. 4A-4C include different views of a schematic representation of an embodiment of a frame 400 that couples a flexible membrane sheet 114 and/or the pin plate 116 to a top plate 110, such as the top plate of FIGS. 3A-3C. As shown, the frame 400 can include a frame body 410 that is shaped and dimensioned to match the top plate 110 and/or pin plate 116. The frame body 412 can include a shelf 412 that is dimensioned to receive the top plate 110. The depth of the shelf 412 can be adjusted by adjusting the height of the side wall 413. The frame body 412 can include one or more end support members 414 and one or more side support members 416 dimensioned to receive the pin plate 116. The location and size and shape of the support members 414, 416 can be varied, and they may be integrated, movable, adjustable, or other configuration.

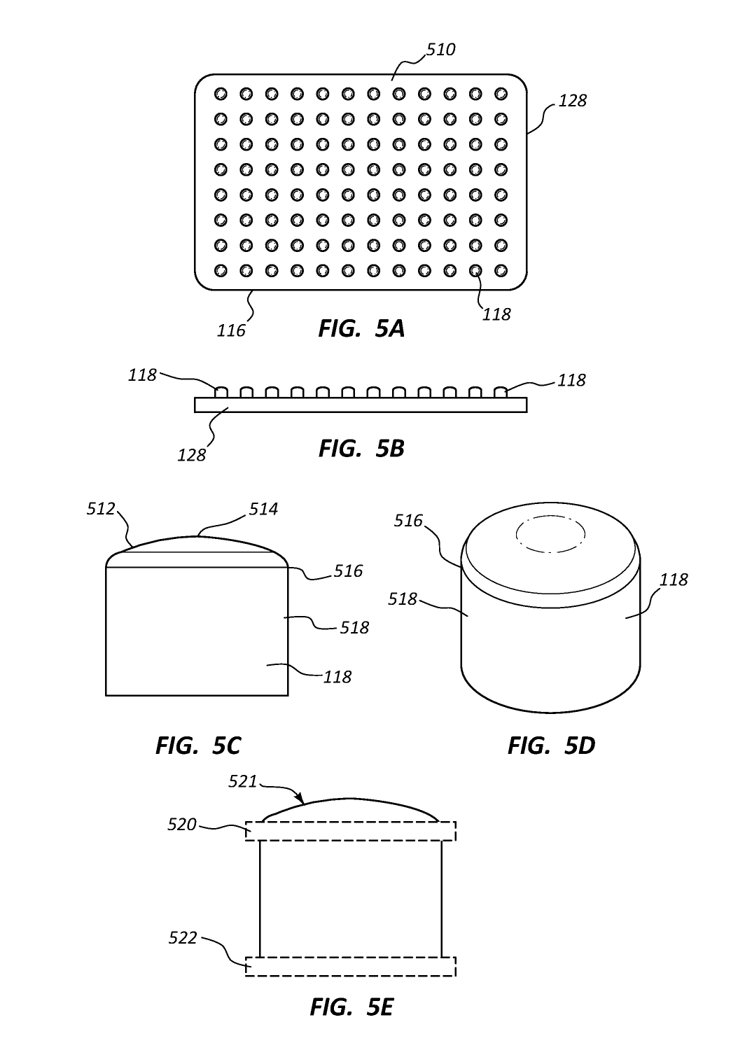

FIGS. 5A-5B include different views of a schematic representation of an embodiment of a pin plate 116 in accordance with the present invention. The pin plate 116 can include the pins 118 as described herein. The pins 118 can be jointly or separately mounted on a pin surface 510 of the pin plate 116. The pin plate 116 can have a side surface that is dimensioned in accordance with the support members 414, 416.

FIGS. 5C-5D include different views of a schematic representation of an embodiment of a pin 118 of the pin plate 116 of FIGS. 5A-5B. The pin, 118 can include a crown 514 with a sloped top surface 512 down to a neck 516. The pin 118 can include a side surface 518 of various height.

FIG. 5E includes a schematic representation of optional embodiments of a movable pin 521 that is movable in a pin plate 116 that has a pin hole 124 in accordance with the present invention. The pin 512 can include a top flange 520 and/or a bottom flange 522, where either flange is optional. The flanges 520, 522 can interact with a pin hole to limit or restrict pin 521 movement. The flanges 520, 522 can keep the pin 521 movably coupled to the pin plate 116.

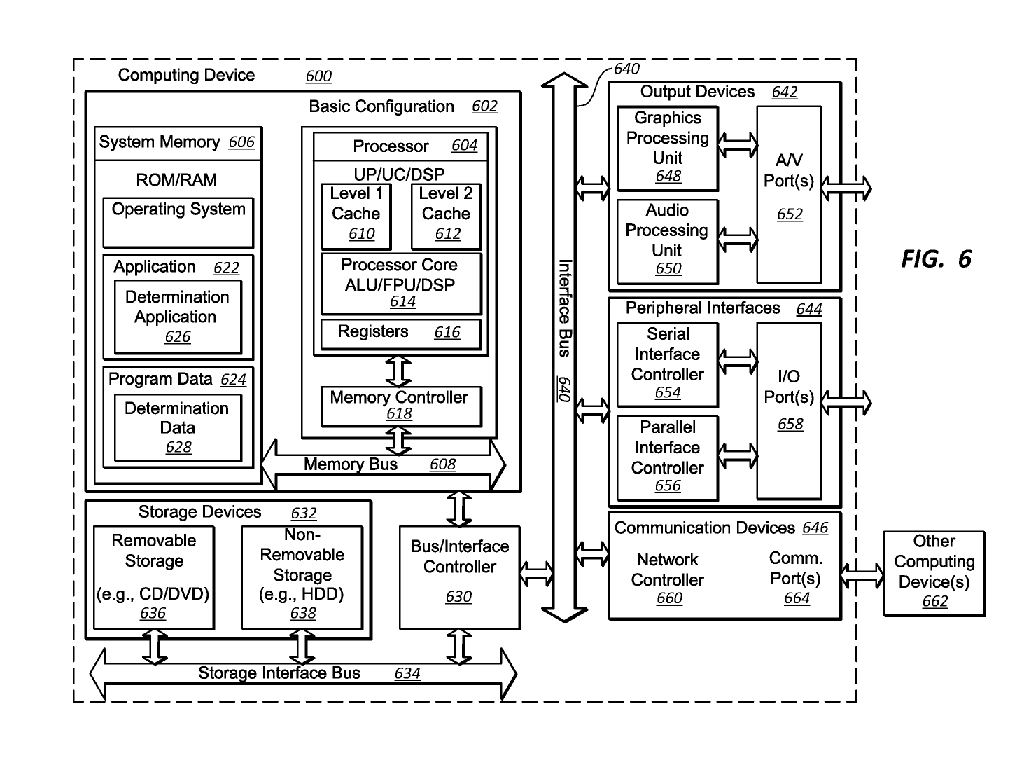

FIG. 6 includes a schematic representation of an embodiment of a computing system or device 600 that can be used to control a plate system in order to perform the mechanically strained cell culture methods and drug screening methods in accordance with the present invention. The computing system or device 600 is illustrated in more detail below.

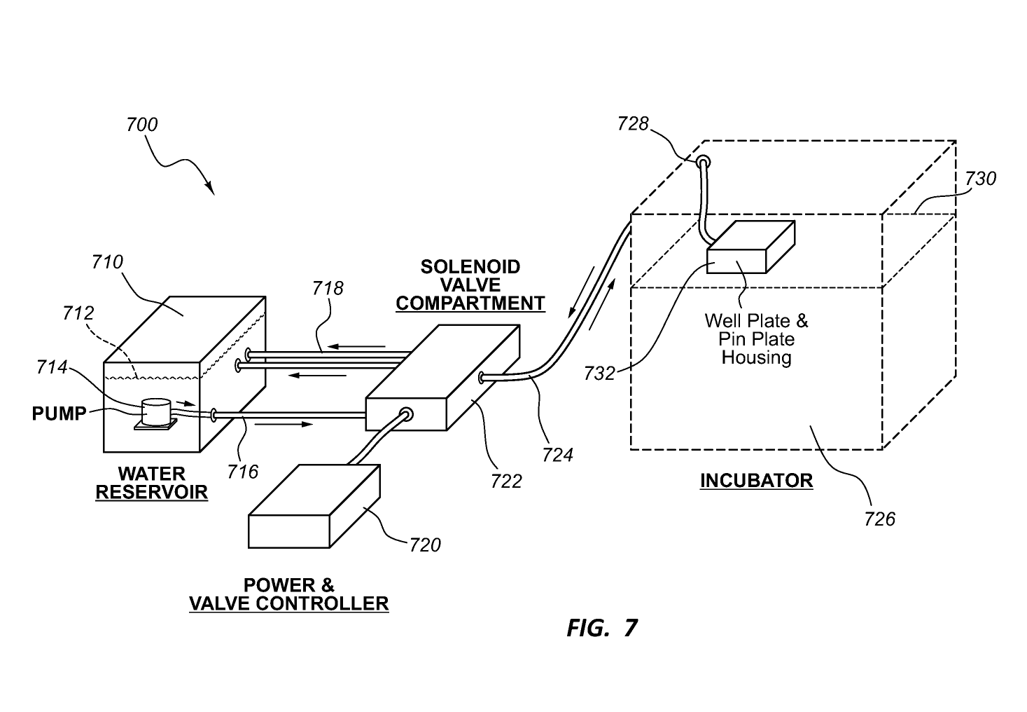

FIG. 7 includes a schematic representation of an embodiment of a system 700 for implementing mechanically strained cell culture methods and/or drug screening with an embodiment of a plate system 732 in accordance with the present invention. The system 700 is shown to include a fluid reservoir 700 having a fluid 712 (e.g., water) with a pump 714 located to pump the fluid 712 through a fluid out line 716 to a solenoid valve device 722. The solenoid valve device 722 can be controlled by a computing device 720. the solenoid valve device 722 can also be fluidly coupled to fluid in lines 718 that allow fluid 712 to flow back into the reservoir 710. The solenoid valve device 722 can also be fluidly coupled to a fluid line 724 that is fluidly coupled to a plate system 732 as described herein where the plate system 732 uses fluidics to move the pins 118 and flexible membrane bottoms 122 of the wells 112. The fluid line 724 enters into an incubator 726 through a port 728 in the incubator 726. The incubator 726 can include a shelf 730 to hold the plate system 732.

In one embodiment, port 728 can be used to introduce a gaseous toxin into the incubator. For example, the gaseous toxins can include cigarette smoke, engine exhaust, or the like.

As described herein, the illustrated plate system 732 can be used to stretch cells in monolayers and in 3D cultures or tissues. The plate system 732 can also be compatible with standard plate readers. The plate system 732 can also be modified so that the pins 118 can push and/or pull the membrane bottoms 122. In order to pull the membrane bottoms 122, and adhesive or other adhering agent can be used to adhere the pins 118. A removable adherence can be favorable, which can be obtained with various pressure-sensitive adhesives, such as polyisobutylenes or silicone adhesives or acrylic adhesives.

The plate system 732 can include a 96-well plate with well 112 having a deformable base 122 capable of producing 5-15% strain over 70% of the functional well surface to enhance the growth of cultured cells.

The plate system 732 can be dimensioned based on standard specifications for existing non-deformable 96-well plates. The well plate 110 can be prepared by being CNC machined from a 12×5×0.5 inch delrin block, then fly-cut and sanded along one face to create a smooth surface. The 96 wells can be drilled with a F-sized bit (φ0.257 inch) in an evenly spaced 8 by 12 hole pattern. Peripheral geometry can be machined to be similar to standard plate geometry so that it can fit in existing standard laboratory equipment. A deformable silicone membrane can be cut from a 12″×12″×0.02″ sheet (Specialty Manufacturing Inc), which was then adhered to a bottom surface of the top plate using 4014 Loctite® Medical Device super glue, and allowed to cure at room temperature with static pressure applied to the membrane-well plate interface. The design allows for the deformation of discrete portions of the membrane upward into the individual wells of the well plate. A small framework can be fixed to the bottom of the plate (using super glue) to support the adhesion of the membrane to the perimeter of the plate and provide adaptation to existing cell plate readers. The framework can be configured and dimensioned to provide a small gap underneath the membrane to the bottom of the plate.

Various manufacturing techniques can be used to prepare plate systems as described herein. For example, the manufacture methods can include injection molded the various components, while the attachment of the membrane and framework would likely be secondary and tertiary manufacturing steps.

In order to deform the well membrane bottoms with desire, defined, or other specified strain, a pin plate having fixed pins can be hydraulically displaced vertically upward into the well membrane bottoms while the well plate is held stationary. The well plate and indenter plate can both be housed in a small rectangular framework, referred to as the frame 400 of FIGS. 4A-4C.

The frame can be prepared from ABS plastic, and dimensioned such that the well plate can be fixed and movement of the pin plate can be restricted to vertical displacement at various heights. The pin plate can also made of rapid prototyped ABS plastic, and can be dimensioned to approximately the same length and width of the well plate. The pin can be cylindrical (e.g., about 4 mm diameter) with a spherical tip (e.g., 4.26 mm diameter) and have a height that rises 3.5 mm from the base to the end of the tip. Each pin can be positioned to align with the center of the wells of the well plate. Mass production of the pin plate may be accomplished from an injection molding, while the frame may be casted.

The hydraulic actuation of the pin plate can be accomplished from a customized fluid bag. The bag can be fitted with a PVC pipe fitting to connect a standard 0.5″ hose. The bag can sit inside the frame below the pin plate. As the bag fills with fluid (e.g., water), the expansion creates a pressure that pushes the pin plate vertically into the membrane bottom of the well plate wells. The pressure is controlled by the amount of time water is allowed to flow into the bag. After adequate pressure is reached to displace the pin plate 2 mm into the membrane, the water is released causing the pressure of the bag to decrease and the indenter plate to drop. All of the previously mentioned components can be located inside of the environmentally controlled incubator. The components that control the fluid can be located outside of the incubator. A reservoir can house both the pump and the fluid. The pump can be a standard aquarium pump that is capable of producing 4 feet of pressure head to the pin plate. While the pump runs constantly, the flow of water can be directed using three normally-closed delrin solenoid valves. The valves can be powered from standard 120 V AC wall power, and controlled using a Basic Stamp 2 controller and circuit. The opening and closing of each valve can direct water to fill the bag, release water to empty the bag, or maintain the level of water in the bag via standard 0.5″ diameter flexible aquarium tubing. This timing sequence is controlled from a custom code written in the BASIC language, which autonomously executes from the controller. A memory device can include a computer program product that includes computer executable instructions for controlling the system described herein to mechanically strain the well membrane bottom during a cell culture to study one or more chemicals.

The plate system can be configured as follows: a 96 well plate; configuring the plate to be compatible with standard plate readers; designing the plate to have dimensions in accordance to the society for biomolecular sciences' standard for the microtiter plates; do not use vacuum to pull the membrane down over a stationary posts; use system to push the pins upwards into the membrane; use liquid (e.g., water) to move the pins up to stretch the membrane and mechanically strain the cells; use any other stepper motor to move the pins; and/or use any mechanical device to move the pin plate.

In one embodiment, the plate system can be configured to apply different strains to each well by having pins of different heights or independently movable. The pin plate can be configured to prepare pins with different heights, such as designed, random, in columns. or rows.

In one embodiment, the invention can include a method for drug screening under mechanical strain that includes: providing one or more cells in vitro; introducing a chemical to the one or more cells; introducing a mechanical strain (e.g., simulated in vivo strain) to the one or more cells in the presence of the chemical; and determining whether or not the chemical has bioactivity with respect to the one or more cells. The one or more cells can be mammalian cells, such as human cells. The one or more cells can form a monolayer or three-dimensional (3D) cell cultures or tissue structures. The one or more cells can be representative of a disease state, such as cancer. The cells can also be normal or healthy cells, and the studies can be done to determine impact of chemical on the normal or healthy cells. The one or more cells can be genetically modified. The one or more cells can include a non-native polynucleotide that encodes for the production of a non-native polypeptide. The one or more cells can be attached to a flexible substrate. The one or more cells can have a confluency of at least about 80% before the chemical is introduced. The one or more cells can be attached to a membrane capable of being stretched, compressed, bent, deformed, fluctuated, and/or oscillated at selected frequencies.

The mechanically strained in vitro screening can include culturing the one or more cells before, during, and/or after the chemical is introduced. The culturing can include standard cell culture techniques. The mechanically strained in vitro screening can include culturing one or more control cells that do not receive the chemical.

The chemical to be tested under mechanical strain in the systems described herein can be any molecule, small or large or simple or complex, natural or synthetic. The chemical can be selected from a small molecule (under 1 kDa), a potential drug, a potential carcinogen, a potential toxin, a potential chemotherapeutic, a medium molecule (between 1 kDA and 10 kDA), a large molecule (over 10 kDa), a polypeptide, a protein or portion thereof, a polynucleotide, a plasmid, an siRNA, or the like. The chemical can have potential bioactivity based on a static (e.g., typical or not mechanically strained) in vitro screening assay. The chemical can be a potential or known toxin. The chemical can be an element, such as a heavy metal.

Also, the mechanically strained in vitro screening can include performing a static (e.g., typical or not mechanically strained) in vitro screening assay with the chemical on one or more cells.

The chemical can be introduced to the one or more cells in one or more concentrations. Also, the chemical can be included in a carrier.

The mechanical strain (e.g., simulated in vivo strain) can cause the one or more cells to move, stretch, compress, bend, distort, fluctuate, oscillate, aggregate or otherwise be modulated by being moved or stretched as described herein. The mechanical strain can be induced by stretching, compressing, bending, deforming, fluctuating, and/or oscillating a flexible substrate to which the one or more cells is attached. The mechanical strain on the one or more cells can be induced by imparting the mechanical strain to a flexible cell culture substrate in one or more wells of a multi-well plate. The mechanical strain can move, stretch, compress, bend, distort, fluctuate, oscillate, aggregate or otherwise physically modulate the one or more cells. The mechanical strain can be the same for the one or more cells, or different from the one or more cells. The mechanical strain can simulate mechanical forces similar to those experienced in normal in vivo tissues of the one or more cells. The mechanical strain can simulate respiration, musculoskeletal strains, gastrointestinal strains, cardiac strains, or other strains of a body.

The one or more cells in an individual well can be mechanically strained with peripheralcells stretching, compressing, bending, deforming, fluctuating, and/or oscillating more than central cells. The pins can be configured so that all cells experience substantially the same stretching strain.

The bioactivity or toxicity of the chemical can be performed as any screening known or developed so as to use mechanical strain as described herein. There are various such screening assays. Accordingly, the screening of the chemical for bioactivity can be conducted under the mechanical strain.

In one embodiment, the present invention can also include a mechanically straining in vitro screening device. Such a device can include: a well plate frame defining one or more cell culture wells; one or more flexible cell culture substrates coupled with the well plate frame; and one or more mechanical straining members associated with the one or more cell culture wells. The device can include one or more mechanisms for mechanically straining the one or more mechanical straining members. However, such a mechanism can be part of a system in which the device is utilized. The one or more mechanical straining members can be configured to be moved up and down or otherwise actuated so as to modulate the flexible cell culture substrate to mechanically strain the one or more cells. The device can include one or more mechanisms configured to actuate the one or more mechanical straining members.

The one or more flexible cell culture substrates can be configured for one or more of: biocompatible for growing the one or more cells; able to withstand cyclic stretching; or transparent to facilitate the imaging of the one or more cells.

The well plate frame can define a number of wells as is common in standard cell culture plates so that the device can be read on a standard plate reader. For example, the device can include 3, 6, 12, 24, 48 or 96 wells. It can be important for the device to include a plurality of wells for experimental purposes. In one aspect, a single well plate may be insufficient due to a lack of being able to have proper control wells and experimental wells.

The device can be configured with the one or more flexible substrates being configured to be capable of being strained (or stretched or compressed) differently from each other. The device can be configured for various wells of rows or columns to be mechanically strained together and/or differently from other wells of other rows or columns.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims.

The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In one embodiment, the present methods can include aspects performed with a computing system. As such, the computing system can include a memory device that has the computer-executable instructions for performing the method. The computer-executable instructions can be part of a computer program product that includes one or more algorithms for performing any of the methods of any of the claims.

In one embodiment, any of the operations, processes, methods, or steps described herein can be implemented as computer-readable instructions stored on a computer-readable medium. The computer-readable instructions can be executed by a processor of a wide range of computing systems from desktop computing systems, portable computing systems, tablet computing systems, hand-held computing systems, and/or any other computing device.

There is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those generally found in data computing/communication and/or network computing/communication systems.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

FIG. 6 shows an example computing device 600 that is arranged to perform any of the computing methods described herein. In a very basic configuration 602, computing device 600 generally includes one or more processors 604 and a system memory 606. A memory bus 608 may be used for communicating between processor 604 and system memory 606.

Depending on the desired configuration, processor 604 may be of any type including but not limited to a microprocessor (μP), a microcontroller (μC), a digital signal processor (DSP), or any combination thereof. Processor 604 may include one more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations memory controller 618 may be an internal part of processor 604.

Depending on the desired configuration, system memory 606 may be of any type including but not limited to volatile memory (such as RAM), non-volatile memory (such as ROM, flash memory, etc.) or any combination thereof. System memory 606 may include an operating system 620, one or more applications 622, and program data 624. Application 622 may include a determination application 626 that is arranged to perform the functions as described herein including those described with respect to methods described herein. Program Data 624 may include determination information 628 that may be useful for analyzing the contamination characteristics provided by the sensor unit 240. In some embodiments, application 622 may be arranged to operate with program data 624 on operating system 620 such that the work performed by untrusted computing nodes can be verified as described herein. This described basic configuration 602 is illustrated in FIG. 6 by those components within the inner dashed line.

Computing device 600 may have additional features or functionality, and additional interfaces to facilitate communications between basic configuration 602 and any required devices and interfaces. For example, a bus/interface controller 630 may be used to facilitate communications between basic configuration 602 and one or more data storage devices 632 via a storage interface bus 634. Data storage devices 632 may be removable storage devices 636, non-removable storage devices 638, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 606, removable storage devices 636 and non-removable storage devices 638 are examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to basic configuration 602 via bus/interface controller 630. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. Computing device 600 may also be implemented as a personal computer including both laptop computer and non-laptop computer configurations. The computing device 600 can also be any type of network computing device. The computing device 600 can also be an automated system as described herein.

The embodiments described herein may include the use of a special purpose or general-purpose computer including various computer hardware or software modules.

Embodiments within the scope of the present invention also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used herein, the term “module” or “component” can refer to software objects or routines that execute on the computing system. The different components, modules, engines, and services described herein may be implemented as objects or processes that execute on the computing system (e.g., as separate threads). While the system and methods described herein are preferably implemented in software, implementations in hardware or a combination of software and hardware are also possible and contemplated. In this description, a “computing entity” may be any computing system as previously defined herein, or any module or combination of modulates running on a computing system.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.

### EXPERIMENTAL

In vitro screening of chemotherapeutic agents is routinely carried out in static monolayer cell cultures. However, drugs administered to patients act in the presence of various dynamic environments in vivo. For example, in lung tumors, mechanical forces are constantly present and do affect the pharmacological response of the lung tissue to a variety of therapeutic agents. It was hypothesized that mechanical forces may affect the response of lung tumors to chemotherapeutic agents, and studied the effects under simulated conditions.

First, the effects of simulated forces that approximate normal respiration were studied on the proliferation and morphology of NCI-H358 (e.g., H385 cells) and A549 cell lines. The effects of the simulated forces were studied with the ability of paclitaxel, doxorubicin, cisplatin, zactima and an experimental drug (i.e., KU-drug) to induce cytotoxicity in both cell lines. Cells were treated with the drugs in the presence or absence of simulated forces (e.g., 20% maximum strain and 15 cycles/minute) that approximate human lung expansion and contraction. Cell proliferation and the effectiveness of the drugs were assessed using a standard exponential cell growth model, and it was determined that mechanical forces significantly reduced the proliferation of both cell lines. Interestingly, mechanical forces also significantly lowered the effectiveness of all drugs except zactima in A549 cells, while in H358 cells, zactima was the only drug that demonstrated an increase in effectiveness due to mechanically applied forces. The results demonstrate that mechanical forces have significant impact on cell survival and Chemotherapeutic efficacy, and may be of significance in engineering improved screening assays for anti-tumor drug discovery.

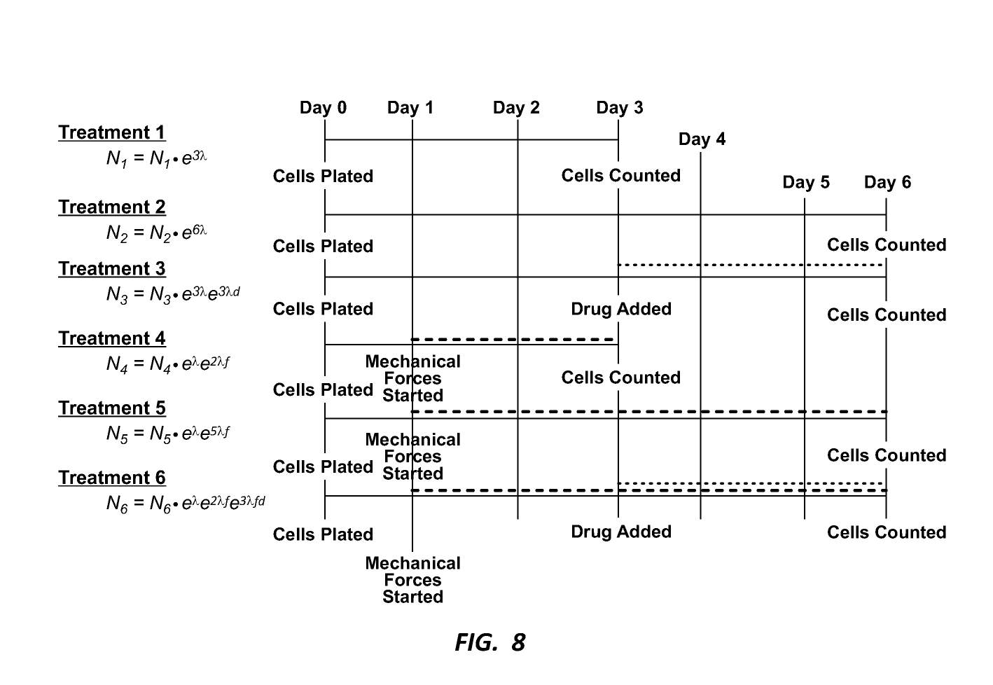

Two human lung cancer cell lines, bronchioalveolar carcinoma (NCI-H358) and epithelial lung carcinoma (A549), and 5 drugs (e.g., paclitaxel, doxorubicin, cisplatin (Sigma Aldrich, St. Louis, Mo.), zactima (LC Laboratories, Woburn, Mass.), and a KU-Drug (experimental drug from the University of Kansas) were used in this study (see Table 1 and FIG. 8). Briefly, following 24 hours of initial seeding, cells were grown under simulated in vivo (e.g., mechanical cyclic stretching) conditions for 2 days in Treatment 4; and for 5 days in Treatments 5 and 6. The cells were exposed to the drugs in the last three days in Treatment 6. Treatments 1, 2 and 3 served as unstretched controls for Treatments 4, 5 and 6, respectively. For each cell line and drug, all treatments were independently repeated at least 3 times on different days, with 3 replicates per Treatments 1 and 4 (n=9) and 5 replicates per all other treatments (n=15).

Table 1 includes a list of investigated chemotherapeutic drugs, their mechanisms of action and the tested concentrations.

TABLE 1

| | | Tested |
|---|---|---|
| | | Concentration |
| Drug | Mechanism of Action | (IC90) |
| Cisplatin | Interacts with and cross-links DNA to | 50 μM |
| | induce apoptosis. | |
| Doxorubicin | Intercalates into DNA and prevents cell | 30 μM |
| | division. | |
| KU-Drug | Inhibits Heat Shock Protein 90 (HSP90). | 50 μM |
| Paclitaxel | Interferes with the normal breakdown of | 50 nM |
| | microtubules during cell division. | |
| Zactima | Tyrosine Kinase Inhibitor that inhibits | 50 μM |
| | Vascular Endothelial Growth Factor | |
| | Receptor (VEGFR) and Epidermal Growth | |
| | Factor Receptor (EGFR) | |

The NCI-H358 (ATCC® CRL-5807) and A549 (ATCC® CCL-185) cell lines were obtained from American Type Culture Collection (ATCC, Manassas, Va.) and cultured in RPMI-1640 (NCI-H358) and HAM's F12K (A549) media containing 10% Fetal Bovine Serum (Sigma Aldrich, St. Louis, Mo.) and 1% Penicillin Streptomycin (Sigma Aldrich). Cells were passaged in 75 cm2 culture flasks at 37° C. in 5% CO2, and trypsinized using TryplE™ Express (Gibco) containing phenol red.

Cells were plated in 6-well Bioflex® plates (Flexcell® International Corporation, Hillsborough, N.C.). Each well of the plate has a flexible elastic membrane coated with a thin layer of type I collagen. Type I collagen was chosen since it more accurately represents the physiology of lung tumor cells in vivo. The Flexcell is not configured for drug studies as described herein, and had to be modified as described herein. Preliminary studies showed that mechanical forces decreased the rate of proliferation of NCI-H358 and A549 cells. Therefore, to have the same number of cells in culture at the time of drug exposure, initial seeding densities for treatments not involving simulated forces (Treatments 1-3) and those involving forces (Treatments 4-6) were adjusted for each cell line. In Treatments 1, 2 and 3, NCI-H358 and A549 cells were plated at a density of 100,000 and 20,000 cells/well in 2 ml of corresponding media, respectively. For treatments involving simulated forces (Treatments 4-6), NCI-H358 and A549 cells were plated at a density of 170,000 and 35,000 cells/well, respectively.

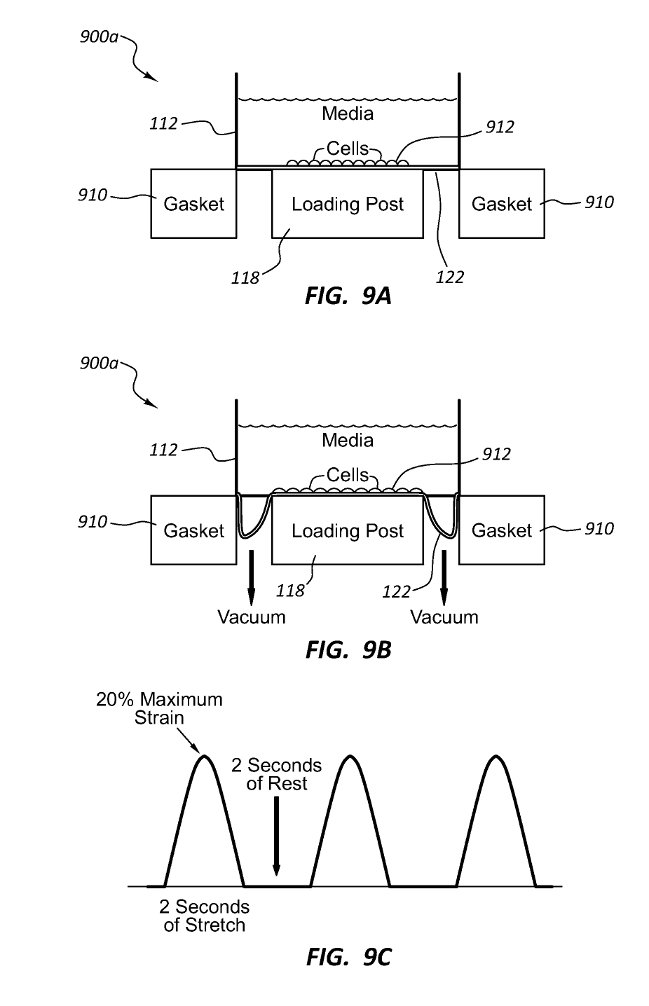

After allowing the cells to adhere to the membrane for 24 hours, the plates assigned for simulated forces were stretched using the FX-4000 Tension plus unit (Flexcell® International Corporation, Hillsborough, N.C.). The Flexcell unit was configured to deliver an alternating positive and negative pressure beneath the cell culture surfaces. Negative pressure pulls the culture surface down over a stationary Delrin post on the baseplate, delivering a uniform biaxial strain to the cells, and subsequent application of positive pressure releases this distension (FIGS. 9A and 9B.) In this manner, the alternating positive and negative pressure simulates forces experienced during respiration. Culture plates experiencing forces were fitted with rubber gaskets and placed on circular loading stations in the baseplate. Cells were subjected to a cyclic strain of 20% maximum strain for 2 seconds, followed by 2 seconds of rest (15 cycles per minute with an average strain of 10% per cycle) in keeping with a normal human respiration rate (2, 27) (FIG. 9C). Cells were preconditioned with mechanical forces for 48 hours, in an incubator at 37° C., 5% CO2 and 95% relative humidity.

After 72 hours of initial seeding, one of the five drugs was added to Treatments 3 and 6 of each cell line. Briefly, the drug was dissolved in Dimethyl Sulfoxide (DMSO) at stock concentration and added to the respective media. An aliquot of 1 ml of drug stock solution was then added to each well containing 2 ml of media (a 1:3 dilution) to get the desired IC90 concentration of the drug (Table 1). The IC90, concentration of the drug that inhibits the growth of 90% of the cells in a static monolayer culture, was used to assure a significant inhibition in the absence of simulated forces. To insure comparability, an aliquot of 1 ml of media without the drug was added to each well in Treatments 2 and 5. After adding the drug or 1 ml of media, the plates from Treatments 5 and 6 were placed back on the baseplate and subjected to the same force profile as before for the remaining 72 hours. Plates from Treatments 2 and 3 (without forces) were placed in the same incubator for the remaining 72 hours.

Cells were trypsinized and counted at the end of day 3 in Treatments 1 and 4, and at the end of day 6 in the other treatments, using a Beckman Coulter Vi-Cell Analyzer (Beckman Coulter, Fullerton, Calif.). To examine changes in morphology, microphotographs of A549 and NCI-H358 cells were taken at the end of Treatments 1, 2, 4 and 5 using an Olympus inverted microscope and a 10× objective.

The standard exponential cell growth model (28) was used to describe the changes in live cell counts after each treatment. According to this model, the cell count (e.g., N) at a particular time (e.g., t), is given by the equation:

N=N*e^{λt}

N* is the initial cell count and λ is the cell growth rate. For each cell line, the following cell growth rates were estimated: λ=growth rate in the absence of forces and drugs, λ_{d}=growth rate in the presence of a particular drug but no forces, λX_{f}=growth rate in the presence of forces but no drugs; and λ_{fd}=growth rate in the presence of forces and a particular drug. The growth rates λ_{d} and λ_{fd} were estimated for each tested drug. Using these growth rates and the exponential growth model, the final cell counts were mathematically modeled for each treatment under various conditions (FIG. 8). For each cell line, a linear regression model that combined the information from the 5 investigated drugs was built using Stata Statistical Software (StataCorp LP, Texas) after log-transforming the six equations listed in FIG. 8. The regression coefficients in the model were the growth rates.

To compare the cytotoxicity of the drugs, we defined the effectiveness of the drug, E, and E_{f} as the percentage reductions in live cell counts in response to a 3-day drug exposure in the absence and presence of simulated forces, respectively. Specifically, for a particular drug (e.g., d), the quantities:

$$E - \frac{{N}_{2} - {N}_{E}}{{N}_{2}} \times 100 \% and {E}_{f} - \frac{{N}_{S} - {N}_{G}}{{N}_{S}} \times 100 \%$$

were used as measures of the effectiveness of the drug in the absence and presence of forces, respectively. These quantities were computed with the formulae

E=(1−e^{-s(λ-λ}^{_{d}}⁾)×100 and E_{f}=(1−e^{-s(λ}^{_{f}}^{-λ}^{_{fd}}⁾)×100.

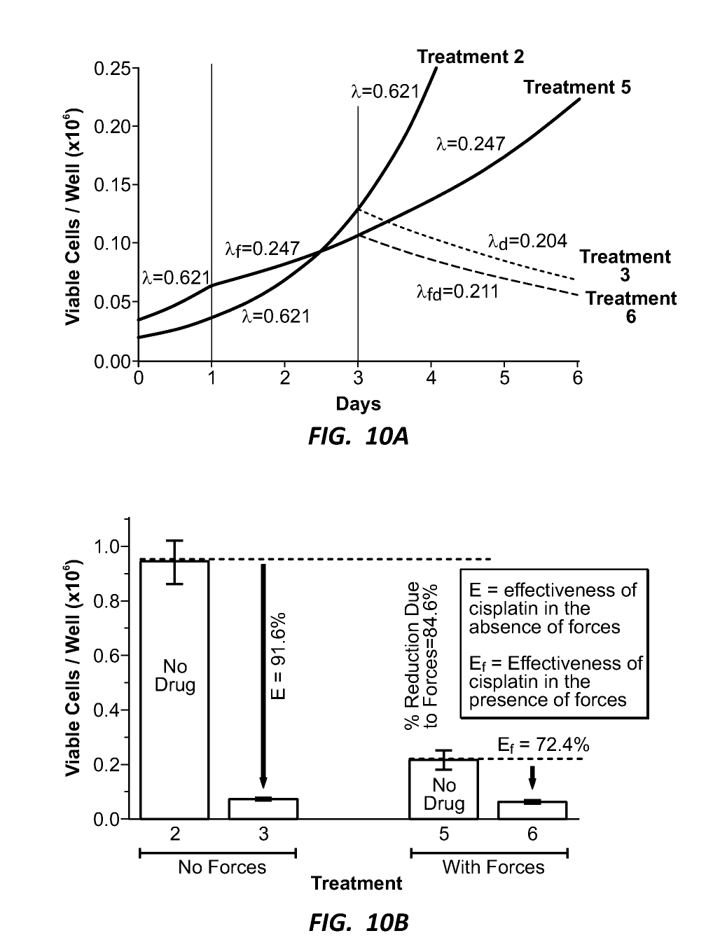

Note that the reduction in live cell number in Treatment 3 is due to cytotoxicity and/or the cytostatic effect of the drugs alone (FIG. 10B). However, the final cell count of Treatment 6 in which drugs are tested in the presence of simulated forces accounts for both increase in cell death due to the cytotoxicity and/or cytostatic effect of the drug and the decrease in live cell population due to mechanical forces (FIG. 10B), the biological mechanisms of which can be quite different. By measuring effectiveness as defined, we compared only the effects of the drugs in the absence and presence of forces. Note that E and E_{f} are independent of cell counts, and E_{f} calculates the effect of the drug alone despite the changes (decrease or increase) in proliferation due to forces. To test the null hypothesis that mechanical forces do not affect drug effectiveness (i.e, E=E_{f}), the significance of the linear contrast:

(λ−λ_{d})−(λ_{f}−λ_{fd})

was examined using Stata's lincom command. This command was also used to compare growth rates in the absence and presence of mechanical forces, and to compute 95% confidence intervals (CIs) for E and E_{f}.

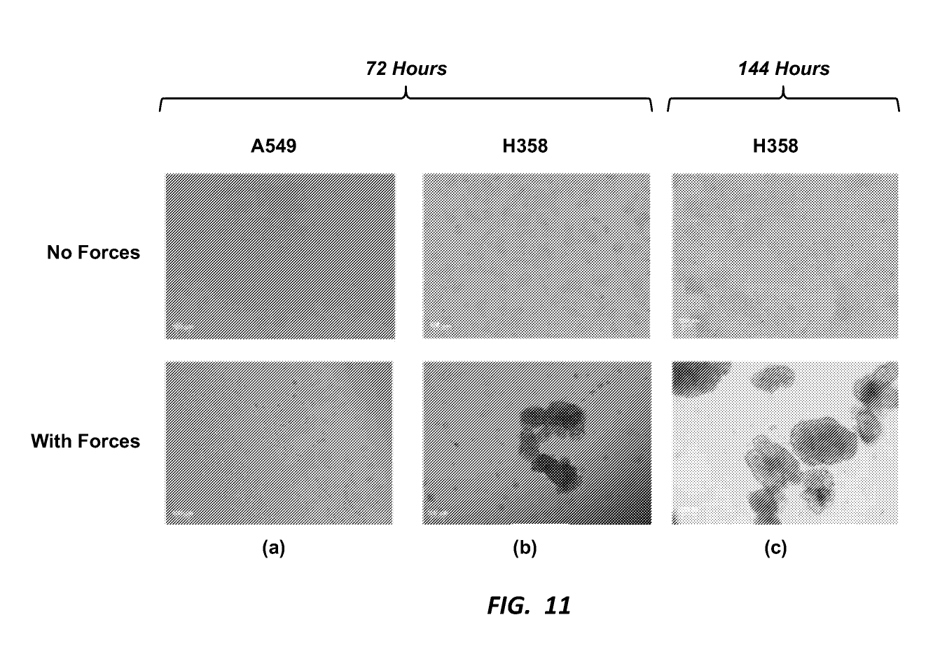

Microscopic examination of NCI-H358 cells showed changes in morphology in response to mechanical forces (FIG. 11). Surprisingly, when subjected to 2 days of simulated in vivo forces (Treatment 4), some of the NCI-H358 cells began to detach from the membrane surface and formed spheroid-like aggregates. After 5 days of simulated forces (Treatment 5), the majority of the cells was detached from the membrane and formed spheroid-like aggregates. All of the detached cells were alive at the end of day 6. In contrast, the NCI-H358 cells that were not subjected to mechanical forces remained attached to the membrane and retained the monolayer morphology at both time points (Treatments 1 and 2). No such changes in morphology were induced by forces in A549 cells which remained attached as a monolayer of cells to the membrane surface of BioFlex Plates (FIG. 11).

In the absence of drugs, mechanical forces significantly decreased the growth rates of both A549 (FIG. 10 column A) and NCI-H358 cells (Table 2). Simulated forces significantly reduced the growth rate of A549 cell line from λ=0.621 per day to λ_{f}=0.247 per day (p<0.001; FIG. 10 column A and Table 2), and that of NCI-H358 cell line from λ=0.402 per day to λ_{f}=0.091 per day (p<0.001; Table 2). The mathematical model estimated that, in the absence of drugs, the percent reduction in cell population due to the application of mechanical forces for 5 days was 84.6% [95% CI, (81.7, 87.0)] for the A549 cell line (FIG. 10 column B), and 78.9% [95% CI, (75.6, 81.7)] for the NCI-H358 cell line. Cell viability measurements of the treatments not involving drugs confirm that the reduction in live cell population is primarily due to a decrease in proliferation or arrested growth and not due to an increase in cell death. Therefore, the results suggest that simulated in vivo forces significantly lower the rate of proliferation of the two tested cell lines in the absence of drugs.

Table 2 shows simulated in vivo forces significantly decreased the rate of proliferation of the two tested cell lines in the absence of drugs, the growth rates of A549 cells when treated with paclitaxel and zactima, and the growth rates of NCI-H358 cells when treated with any of the investigated drug. Note that a negative growth rate represents a reduction in total viable cell count with time. Up arrow indicates the statistically significant increase, down arrow indicates the decrease and horizontal arrows indicate no change in growth rates.

TABLE 2

| | Growth Rates^{a} | | | | | |
|---|---|---|---|---|---|---|
| | A549 | | | H358 | | |
| | | | Change in | | | Change in |
| | Without | | Growth | Without | | Growth |
| Treatment | forces | With forces | Rate^{b} | Forces | With forces | Rate^{b} |
| No Drug | 0.621 | 0.247 | ↓ | 0.402 | 0.091 | ↓ |
| | (0.602, 0.641) | (0.222, 0.272) | (p < 0.001) | (0.391, 0.412) | (0.065, 0.117) | (p < 0.001) |
| [symbol] | −0.204 | −0.211 | [symbol] | −0.027 | −0.233 | ↓ |
| | (−0.302, −0.105) | (−0.309, −0.113) | (p = 0.09) | (−0.080, 0.026) | (−0.339, −0.126) | (p = 0.001) |
| Paclitaxel | 0.133 | −0.038 | ↓ | −0.005 | −0.276 | ↓ |
| | (0.035, 0.232) | (−0.136, 0.060) | (p = 0.02) | (−0.058, 0.048) | (−0.379, −0.174) | (p < 0.001) |
| Doxorubicin | −0.170 | −0.268 | [symbol] | −0.031 | −0.357 | ↓ |
| | (−0.268, −0.071) | (−0.367, −0.170) | (p = 0.2) | (−0.084, 0.022) | (−0.460, −0.254) | (p < 0.001) |
| KU-Drug | −0.390 | −0.464 | [symbol] | −0.105 | −0.337 | ↓ |
| | (−0.489, −0.291) | (−0.562, −0.366) | (p = 0.3) | (−0.158, −0.052) | (−0.440, −0.234) | (p < 0.001) |
| [symbol] | −0.055 | −0.398 | ↓ | −0.163 | −0.833 | ↓ |
| | (−0.141, 0.031) | (−0.486, −0.311) | (p < 0.001) | (−0.209, −0.117) | (−0.927, −0.739) | (p < 0.001) |

When treated with paclitaxel or zactima, mechanical forces significantly decreased the growth rates of A549 cells (Table 2). For instance, in the absence of mechanical forces, the growth rate of A549 cells when treated with Paclitaxel was λ_{d}=0.133 per day; after applying forces the growth rate decreased to λ_{fd}=−0.038 per day (p=0.02). Note that a negative growth rate represents a reduction in total live cell count with time. However, no significant effects of mechanical forces on A549 growth rates were observed after administering Cisplatin (FIG. 10 column A), Doxorubicin or KU-Drug (Table 2). FIG. 10 column A shows the changes in the growth rate of A549 cells when treated with Cisplatin and/or subjected to mechanical forces. In contrast, the growth rates of NCI-H358 cells were significantly decreased by simulated in vivo forces when treated with any of the investigated drugs (Table 2).

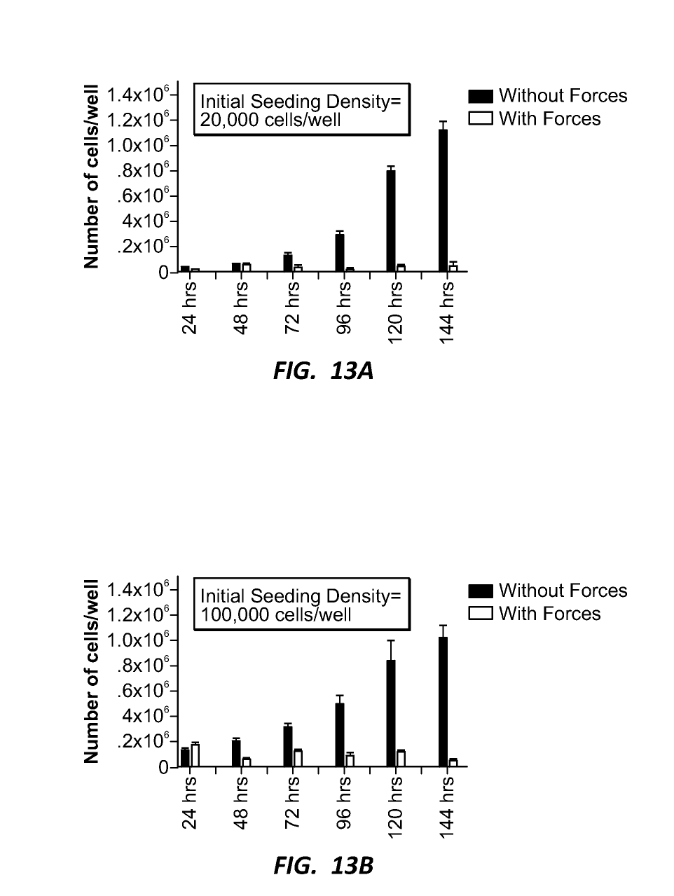

FIGS. 13A-13B shows the number of viable cells with or without mechanical strain forces for cells at an initial seeding of cells up to 144 hours in H358 and A549 cells for the initial seeding densities of 100 000 cells/well for H358 and 20 000 cells/well for A549 cells.

In A549 cell line, mechanical forces significantly decreased the effectiveness of all the investigated drugs except Zactima (Table 3). For instance, in the absence of forces, the effectiveness of Cisplatin was 91.6% [95% CI, (88.5, 93.8)] suggesting that Cisplatin killed 91.6% of A549 cells during the 3-day drug treatment (FIG. 10 column B). However, Cisplatin killed a significantly lower percentage of cells, with an effectiveness of 74.7% [95% CI, (65.4, 81.6)] (p<0.001) in the presence of simulated in vivo forces (FIG. 10 column B). In contrast, for the NCI-H358 cell line, the effectiveness of Cisplatin, Paclitaxel and KU-Drug was reduced by simulated forces, but this reduction was not significant; and the effectiveness of Doxorubicin remained essentially unchanged by forces (E=72.7% and E_{f}=73.9%, p=0.8; Table 3). Notably, for the NCI-H358 cell line, forces significantly increased the effectiveness of Zactima (E=81.6% vs. E_{f}=93.7%, p<0.001).

Table 3 shows mechanical forces significantly decreased the effectiveness of Cisplatin, Paclitaxel, Doxorubicin and KU-Drug when tested in A549 cell line and increased the effectiveness of Zactima when tested in NCI-H358 cell lines.

TABLE 3

| | Effectiveness of The Drug^{a,b} | | | | | |
|---|---|---|---|---|---|---|
| | A549 | | | H358 | | |
| | | With | Change in | | With | Change in |
| | Without | forces | effectivness | Without | forces | effectiveness |
| Drug | forces (E) | (E_{f}) | due to forces^{b} | forces (E) | (E_{f}) | due to forces^{b} |
| [symbol] | 91.6% | 74.7% | ↓ | 72.4% | 62.1% | [symbol] |
| | (88.5, 93.8) | (65.4, 81.6) | (p < 0.001) | (67.3, 76.6) | (46.8, 73.1) | (p = 0.1) |
| Paclitaxel | 76.9% | 57.5% | ↓ | 70.5% | 66.8% | [symbol] |
| | (68.4, 83.1) | (41.8, 69.0) | (p = 0.008) | (65.1, 75.1) | (53.8, 76.1) | (p = 0.5) |
| Doxorubicin | 90.7% | 78.7% | ↓ | 72.7% | 73.9% | [symbol] |
| | (87.3, 93.2) | (70.8, 84.5) | (p < 0.001) | (67.7, 76.9) | (63.7, 81.3) | (p = 0.8) |
| KU-Drug | 95.2% | 88.2% | ↓ | 78.2% | 72.3% | [symbol] |
| | (93.4, 96.5) | (83.8, 91.4) | (p < 0.001) | (74.2, 81.5) | (61.4, 80.1) | (p = 0.2) |
| [symbol] | 86.8% | 85.6% | [symbol] | 81.6% | 93.7% | ↑ |
| | (82.6, 90.0) | (80.8, 89.2) | (p = 0.7) | (78.7, 84.2) | (91.5, 95.4) | (p < 0.001) |

Effects of mechanical forces on engineered 3-dimensional tumor tissues were also studied, An experiment was conducted with paclitaxel, which was tested in the presence and absence of mechanical forces in H358 cells (bronchioalveolar carcinoma) grown as 3D tissues in type 1 rat tail collagen scaffold. The cells were seeded at a density of 4×10⁶ cells per well in 6-well Bioflex collagen I plates and cultured in PRMI 1640 medium The forces that were tested were configured to mimic normal respiration. After 24 hours of seeding, cells were stretched from 0% to a maximum of 20% elongation for 2 seconds followed by 2 seconds of rest continuously for 4 days. Control group included cells that were plated in collagen at the same densities and incubated for 5 days without mechanical forces. FIG. 9C shows a similar mechanical strain period of straining and relaxation. As a note, a computing system similar to FIG. 6 can be used to control an experiment system in order to provide predetermined cycles of mechanical strain and relaxation.

Following 24 hours of stetching, paclitaxel was added to H358 cells in ten 1:3 dilutions with starting concentrations 100 μM. Cells were exposed to these drugs for 72 hours, and the viability was assessed using MTS assay (CellTiter 96 AQueous One Solution Cell Proliferation Reagent, Promega, Madison, Wis.). Dose response curves were plotted and corresponding IC₅₀ values (concentration of the drug at which 50% of the cells are inhibited) were calculated for stretched and un-stretched conditions using Graphpad Prism software.

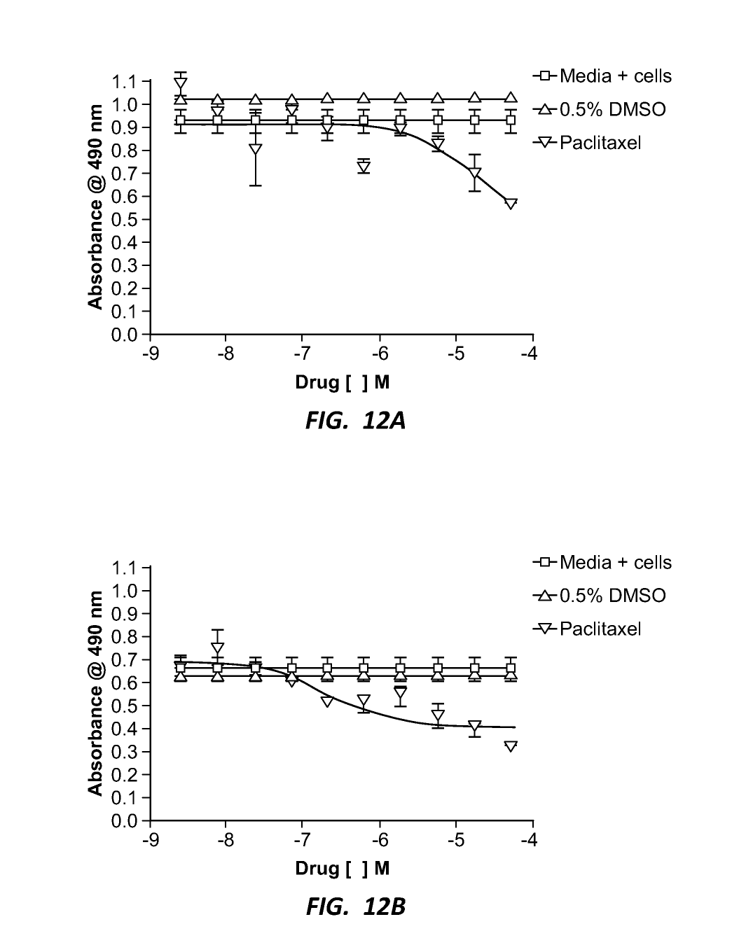

Initial 3D culture antiproliferative assays comparing the treatment of H358 lung cancer cells in the presence and absence of mechanical forces demonstrated differences in IC₅₀ concentrations and efficacy when treated with paclitaxel (FIGS. 12A-12B). In the absence of physiologically relevant lung forces (mechanical stress), paclitaxel was almost two orders of magnitude less potent (IC₅₀=22.6 μM) compared to cells experiencing mechanical stress (IC₅₀=0.2 μM). These data suggest that there are distinct differences in the response of lung cancer cells under physiologically relevant mechanical forces that can impact the effect of treatment. The results also suggest that the current screening process can be improved by incorporating simulated physiologically relevant mechanical forces.

To mimic the effects of normal human respiration we used a force profile with 20% maximum strain at 15 cycles per minute (cpm) (FIG. 9C). Although the actual percentage of distension of lung cells during breathing is not definitively known, in general, strain amplitudes corresponding to changes in surface area less than 25-30% have been shown to be non-injurious and reflective of physiological stretch that occurs at less than total lung capacity, while surface area changes over 37% for longer durations have been shown to cause cell damage.

As shown by Table 3, in the NCI-H358 cell line, only Zactima demonstrated a significant change (an increase) in effectiveness in the presence of simulated forces. Zactima is a vascular endothelial growth factor receptor (VEGFR) tyrosine kinase inhibitor and also targets the phosphorylation of EGFR. Although the effect of forces on the VEGFR expression of these two cell lines is not known, preliminary studies (data not shown) show a decrease in EGFR expression after 2 and 5 days of simulated forces in NCI-H358 cells. This could be a possible explanation for the increased effectiveness of Zactima in NCI-H358 cells. Little or no changes were observed in the effectiveness of Cisplatin, Paclitaxel, Doxorubicin or KU-Drug with the application of mechanical forces.

Additionally, an experiment was conducted to determine the effects of anti-tumor drugs on lung cancer cell lines using a 96 well plate configured in accordance with the present invention. The experiments were conducted with and without the mechanical forces. Plates were sprayed with 70% ethanol and taken into the bio-safety hood. 50 μl of 70% ethanol was added to each well and kept it in the bio-safety hood under UV lamp for one hour to be sterilized. About 50 mg/ml collagen solution was made by dissolving in 0.02 M acetic acid. About 100 μl of this solution was added to each well and kept in the room temperature for 1 hour. After one hour, the solution was aspirated from the plates. The plates were washed three times with 100 μl of PBS to remove the acid. Human lung cancer cell lines, bronchioalveolar carcinoma NCI-H358 (ATCC CRL 5807) and epithelial lung carcinoma A549 (ATCC CCL-185) were used in this experiment. Anti-tumor drugs Doxorubicin, Paclitaxel and Zactima were tested in this experiment. (Table 1—Mechanism of action and tested concentration.) Cells were cultured in 75 cc flask using HAM's F12K (A549) and RPMI1640 media (H358) containing 10% Fetal Bovine Serum (Sigma Aldrich) and 1% Penicillin Streptomycin (Sigma Aldrich) for 48 hours at 37° C. in 5% CO2 until they achieved 85% confluency. On the day of experiment, media from the confluent cells were aspirated and cells were trypsinized using 3 ml of tripLE (Invitrogen) to detach from the flask. The detached cells were mixed with 10 ml of fresh respective media and aspirated into labeled 15 ml conical tubes. Cells were centrifuged at 1000 rpm for 5 minutes and supernatant were aspirated out and the cell pellet was mixed with new 10 ml of respective media. One ml of this cells suspension was counted using Vi-Cell counter (Beckman Coulter) to determine the number of viable cells. A549 cells were seeded at a density of 2000 cells in 100 μl/well in the bottom half wells of two prototype 96-well plates and H358 cells were seeded at 3000 cells in 100 μl/well) in the top half wells of the 96 well plates.

After allowing the cells to adhere to the flexible membrane of the 96 well plate for 24 hours, the loading pins and the system described in FIG. 7 to implement mechanical strain were used to deliver 20% maximum strain for 2 seconds to the plate assigned for mechanical forces (+M Group), followed by 2 seconds of rest (15 cycles per minute with an average strain of 10% per cycle) in keeping with a normal human respiration.

In the two part experiment, first the effect of mechanical forces on the proliferation of A549 and H358 cells were assessed after 3 days of mechanical stretching by two different techniques; 1) counting the viable cells on the ViCell counter and 2) using MTS assay.

In a parallel experiments, the cells were treated with anti-tumor drug after 72 hours of initial seeding (after 48 hours of mechanical stretching). The drug was dissolved in dimethyl sulfoxide (DMSO) at stock concentration and added to the respective media. An aliquot of 50 μl of drug stock solution was added to the well containing 100 μl of media (a 1:3 dilution) in a drug plate to get the desired IC₉₀ concentration of the drug (Table 1). The IC₉₀ concentrations of the drugs were then added to prototype plate.

The +M Group was subjected to continuous mechanical stretching while the −M Group plate was placed in the same incubator for additional 24 hours.

In order to quantify viability, 6 wells per treatment group for ViCell counter and 6 wells per condition for MTS were assigned to determine the cell viability. Using Vi-Cell counter, assigned wells were filled with extra 210 μl of media to keep the volume constant in all the wells. Media from the assigned wells were transferred into a 24-well plate, and the well location was recorded. About 50 μl of trypLE was added to each well and allowed to trypsinize for a maximum of ten minutes. A small amount of media from the 24-well plate was transferred back into corresponding wells of the prototype plate to collect detached cells.

Media and cells were transferred back into the corresponding well of the 24-well plate. All wells were brought to a final volume of 1.25 mL with PBS and mixed well. About 1 ml of this cell suspension was transferred to ViCell counter to determine the number of viable cells. MTS y assay (CellTiter 96 AQueous One Solution Cell Proliferation Reagent, Promega, Madison, Wis.) was also used to study cell viability. Beginning with the −M group plate, 30 μL of MTS was added to the assigned wells. The timing of the reaction was started as soon as all wells are filled with MTS dye.

The prototype plate was read using the Biotek plate reader at the wavelength of 490 nm until the media only wells absorbance reached 1.0. For the +M group plate, 30 ul of MTS was added to the assigned wells and kept in the incubator for the same amount of time that required for the −M group to develop and read at the same wavelength.

Tables 4 and 5 provide results to the forgoing experimental protocol.

TABLE 4

| | Viable Cell Count after 24 hours of drug exposure | | | | | |
|---|---|---|---|---|---|---|
| | A549 | | | H358 | | |
| | | | Change in | | | Change in |
| | | With | viable cell | | With | viable cell |
| | Without | forces | count due to | Without | forces | count due to |
| Drug | forces (E) | (E_{f}) | forces | forces (E) | (E_{f}) | forces |
| Paclitaxel | 33125.0 | 11875.0 | [symbol] | 21250.0 | 9687.5 | [symbol] |
| | (4635.124) | (2771.695) | (p = 0.0386) | (4704.829) | (786.441) | (p = 0.1188) |
| Doxorubicin | 30500.0 | 11750.0 | [symbol] | 17750.0 | 7500.0 | [symbol] |
| | (4340.939) | (2113.942) | (p = 0.0097) | (728.869) | (1369.306) | (p = 0.0021) |
| [symbol] | 23333.3 | 15625.0 | [symbol] | 22000.0 | 11000.0 | [symbol] |
| | (5210.833) | (2971.216) | (p = 0.1372) | (3905.125) | (1145.644) | (p = 0.0493) |

TABLE 5

| | MTS absorbance after 24 hours of drug exposure | | | | | |
|---|---|---|---|---|---|---|
| | A549 | | | H358 | | |
| | | With | Change in | | With | Change in |
| | Without | forces | absorbance | Without | forces | absorbance |
| Drug | forces (E) | (E_{f}) | due to forces | forces (E) | (E_{f}) | due to forces |
| Paclitaxel | 0.567 | 0.293 | [symbol] | 0.522 | 0.345 | [symbol] |
| | (0.057) | (0.015) | (p = 0.1124) | (0.018) | (0.014) | (p = 0.0053) |
| Doxorubicin | 0.697 | 0.430 | [symbol] | 0.629 | 0.488 | [symbol] |
| | (0.046) | (0.030) | (p = 0.0192) | (0.018) | (0.016) | (p = 0.0868) |
| Zactima | 0.495 | 0.307 | [symbol] | 0.536 | 0.368 | [symbol] |
| | (0.012) | (0.011) | (p = 0.0005) | (0.016) | (0.031) | (p = 0.0033) |

Three days of mechanical forces significantly reduced the proliferation of A549 cells by 52.81% (p=0.0026).

Statistically significant reduction in the viable cell count was observed for the Paclitaxel and Doxorubicin treatments in A549 cells with the application of mechanical forces. Zactima's ability to inhibit the cancer growth remained unaffected with forces in A549 cells (Table 4).

Despite the duration of the drug exposure (24 hours in this experiment versus 72 hours in previous experiment), these findings (changes in proliferation of A549 cells and cytotoxicity of Paclitaxel and Doxorubicin in response to forces) are similar to the findings we previously obtained using FX-4000 Tension plus unit (Flexcell® International Corporation, Hillsborough, N.C.).

Similarly, mechanical forces significantly lowered the proliferation of H358 cells by 51.52% (p=0.0053).

The cytotoxicity of Paclitaxel's in H358 cells remained unaffected with forces when assessed using the viable cell count (Table 4). These finding are also consistent with the previous findings obtained with the FX-4000 Tension plus unit (Flexcell® International Corporation, Hillsborough, N.C.).

Contrary to the previous experiment (with Flexcell instrument), results from this experiment show a decrease in the ability of Doxorubicin and Zactima to induce cytotoxicity in H358 cells. This inconsistency may be attributed to the different durations of the experiments and plate geometry considerations to be optimized as the prototype plate is refined. Additionally, we have previously demonstrated using Flexcell instrument that some of the H358 cells began to detach from the membrane after 2 days of mechanical forces and the majority of the cells detached after 3 additional days of mechanical forces. The fact that these cells detach from the growth surface and remain suspended in media in clumps of growing aggregates when mechanical forces are applied, could possibly protect them from further changes mediated by mechanical forces. However, the H358 cells were subjected to mechanical forces for only 3 days in the current experiment. This short period of mechanical stimulation could have altered the H358 cells' response to Doxorubicin and Zactima.

We also measured the proliferation of A549 cells and H358 cells, and the cytotoxicity of the investigated drugs in these two cell lines using MTS assay in our prototype plate using the standard Biomek plate reader to demonstrate compatibility (Table 5).

With the applied forces, similar reductions in proliferation were detected in A549 and H358 cells [29.82% (p=0.0006) and 43.77% (p=0.0134) reduction, respectively] using MTS assay.

Three of the six comparisons (Doxorubicin in A549 cells, Doxorubicin in H358 cells and Zactima in A549 cells) to determine the effect of the treatment in the presence and absence of mechanical forces produced identical results as obtained by counting the viable cells using Vi-cell counter. The remaining three comparisons (Paclitaxel in A549, Paclitaxel in H358 and Zactima in H358) offered different results from those obtained by counting the viable cells. Our results suggest that the inventive 96 well plate is compatible with standard drug screening tools (e.g., plate readers, anti-proliferative assays, etc) can be used in high throughput screening.

1. A method for in vitro drug screening, the method comprising:
providing one or more cells in vitro;
introducing a chemical to the one or more cells;
introducing a mechanical strain to the one or more cells in the presence of the chemical; and
determining whether or not the chemical has bioactivity with respect to the one or more cells under the mechanical strain.

2. The method of claim 1, wherein the one or more cells are one or more of the following: mammalian cells; cells with substantially normal physiology; form a monolayer; form 3D spheroids; form tissues; form 3D cell cultures; representative of a disease state; representative of cancer; genetically modified; including a non-native polynucleotide that encodes for the production of a non-native polypeptide; a combination of cell types; or a single cell type.

3.-10. (canceled)

11. The method of claim 1, wherein the one or more cells are attached to a flexible cell culture substrate.

12. (canceled)

13. The method of claim 1, wherein the one or more cells are attached to a membrane capable of stretching, compressing, bending, deforming, fluctuating, and/or oscillating so as to cause the one or more cells to stretch, compress, bend, deform, fluctuate, and/or oscillate or otherwise be physically modulated.

14.-15. (canceled)

16. The method of claim 1, further comprising culturing one or more control cells that do not receive the chemical and/or that do not receive the mechanical strain.

17.-18. (canceled)

19. The method of claim 1, further comprising performing a static in vitro screening assay with the chemical on one or more cells.

20.-28. (canceled)

29. (canceled)

33.-35. (canceled)

36. A dynamic in vitro screening device comprising:
a top plate defining one or more cell culture wells;
one or more flexible cell culture substrates operably coupled with the top plate to form fluid tight cell culture well bottoms; and
a pin plate under the top plate with the one or more flexible cell culture substrates therebetween, the pin plate having one or more pin members associated with the one or more cell culture wells.

37. The device of claim 36, further comprising one or more mechanisms for actuating the one or more pin members together or separately.

38.-41. (canceled)

42. The device of claim 36, wherein only one flexible substrate is associated with the top plate.

43. The device of any one of claim 36, wherein the flexible substrate is a membrane.

44.-45. (canceled)

46. The device of claim 36, wherein the device is configured with the one or more flexible substrates being configured to be capable of being strained differently from each other.

47. The device of claim 36, wherein the device is configured for various wells of rows or columns to be mechanically strained together and/or differently from other wells of other rows or columns.

48. The device of any one of claim 36, wherein the one or more pins are cylindrical pins with rounded heads to provide bi-axial strain to the cells.

49. (canceled)

50. The device of claim 36, further comprising a mechanism to oscillate the pin plate having fixed pins.

51. The device of claim 50, wherein the mechanism includes: a linear actuator; an electromagnetic system; or a pneumatic system.

52.-55. (canceled)

56. The device of claim 36, wherein the flexible membrane is transparent.

57. The device of claim 36, wherein the device is configured to provide tension and/or compression to cells in the wells.

58. A system for in vitro screening, the system comprising:
a device as in claim 36; and
a device that is configured to actuate the one or more mechanical straining members.

59. (canceled)

60. A method for in vitro drug screening, the method comprising:
providing a three dimensional cell culture in vitro, said three dimensional cell culture having a plurality of cellular layers;
introducing a chemical to the three dimensional cell culture; and
determining whether or not the chemical has bioactivity with respect to the cells of the three dimensional cell culture.

61. (canceled)
